(19) United States
(12) Patent Application Publication
(10) Pub. No.: US 2006/0090755 A1
(43) Pub. Date: May 4, 2006

(54) Respirator fluid feed-line system and method of use

(76) Inventor: Nickolas R. King, Brigham City, UT (US)

Correspondence Address: Brent T. Winder;Jones Waldo Holbrook & McDonough, Suite 1500, 170 South Main Street, Salt Lake City, UT, 84101-1644, US

(21) Appl. No.: 10/980,399

(22) Filed: Nov. 3, 2004

(51) Int. Cl. A61M 15/00

(52) U.S. Cl. 128/202.15

(57) ABSTRACT

A respirator fluid feed-line system that includes a respirator having a first side and a second side opposite the first side. A feed-line connector is located on the first side; and an inside fluid feed-line is located on the second side. The inside fluid feed-line is in communication with the feed-line connector on a first end, and has a free second end capable of being inserted into a user's mouth. The system also can include a fluid receptacle having an opening through which air can pass. The system can further include a filter, in communication with the opening, and positioned substantially between the opening and an outside air source. The filter can include a HEPA cartridge and a spring valve, such that when air is passing into the receptacle, the spring valve is opened, and when air is not passing into the receptacle, the spring valve is closed. An outside fluid feed-line, having a third and a fourth end, can also be included, wherein the third end is capable of being coupled to the feed line connector, and the fourth end is capable of being coupled to the fluid receptacle such that the fluid receptacle, the outside fluid feed-line and the inside fluid feed-line are in communication with each other.

### BACKGROUND

There are a number of occupations in which an individual is required to wear a respirator, and other similar sorts of protective gear, for extended periods of time. For example, in the asbestos removal profession, a worker is required by OSHA regulation to wear a respirator throughout the day, with only intermittent breaks that permit the respirator's removal.

The asbestos worker is often working in environments without climate or temperature control of any sort, and thus in summer months, or in warm climates, such workers are particularly prone to dehydration and/or heat exhaustion. This problem is further exacerbated by the fact that the worker, in addition to wearing a respirator, is also wearing other heavy protective gear.

From the employer's perspective, this problem can also be financially straining as workers removing their respirators while on a job can cause hefty fines to be levied against the employer.

To alleviate each of these problems, as well as others not specifically mentioned, Applicant has devised the present invention.

### SUMMARY

The present invention is a respirator fluid feed-line system and method of use. The invention includes a respirator having a first side and a second side opposite the first side. A feed-line connector is located on the first side; and an inside fluid feed-line is located on the second side. The inside fluid feed-line is in communication with the feed-line connector on a first end, and has a free second end capable of being inserted into a user's mouth. The system also can include a fluid receptacle having an opening through which air can pass. The system can further include a filter, in communication with the opening, and positioned substantially between the opening and an outside air source. The filter can include a HEPA cartridge and a spring valve, such that when air is passing into the receptacle, the spring valve is opened, and when air is not passing into the receptacle, the spring valve is closed. An outside fluid feed-line, having a third and a fourth end, can also be included, wherein the third end is capable of being coupled to the feed line connector, and the fourth end is capable of being coupled to the fluid receptacle such that the fluid receptacle, the outside fluid feed-line and the inside fluid feed-line are in communication with each other. The system can include a threaded female fitting on the first end of the outside fluid feed-line; and a corresponding threaded male fitting on the feed-line connector. The inside feed-line can also include a bite valve. The fluid receptacle can also include an insulated cover.

A method of providing a fluid to a user of a respirator is also disclosed. The method comprises the steps of providing a respirator having a first side and a second side opposite the first side; a feed-line connector on the first side; and an inside fluid feed-line on the second side, in communication with the feed-line connector on a first end, and having a free second end capable of being inserted into a user's mouth. A third end of an outside fluid feed-line can be coupled to the feed line connector, and a fourth end of the outside fluid feed-line can be coupled to the fluid receptacle such that the fluid receptacle, the outside fluid feed-line and the inside fluid feed-line are in communication with each other. The free second end of the inside fluid feed-line can then be inserted into the user's mouth; who can then provide suction at the free second end, thereby creating a negative pressure in the inside fluid feed-line. By so acting, the fluid is conveyed from the receptacle, through the outside fluid feed-line, through the feed-line connector, through the inside fluid feed-line and into the user's mouth.

### BRIEF DESCRIPTION OF THE DRAWINGS

FIG. 1 shows a front-side view of a respirator equipped with a fluid feed-line attachment according to one embodiment of the present invention.

FIG. 2 shows an inside view of a respirator equipped with a fluid feed-line attachment according to one embodiment of the present invention.

FIG. 3 shows a fluid receptacle suitable for use with a fluid feed-line attachment according to one embodiment of the present invention.

FIG. 4 shows a fluid receptacle lid according to one embodiment of the present invention.

FIG. 5(a) is a side-view of the face-piece showing one orientation of a fluid feed-line connector, according to one embodiment of the present invention.

FIG. 5(b) is a side-view of the face-piece showing another orientation of a fluid feed-line connector, according to yet another embodiment of the present invention.

### DETAILED DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. These drawings depict only typical embodiments of the invention and are not therefore to be considered to be limiting of its scope.

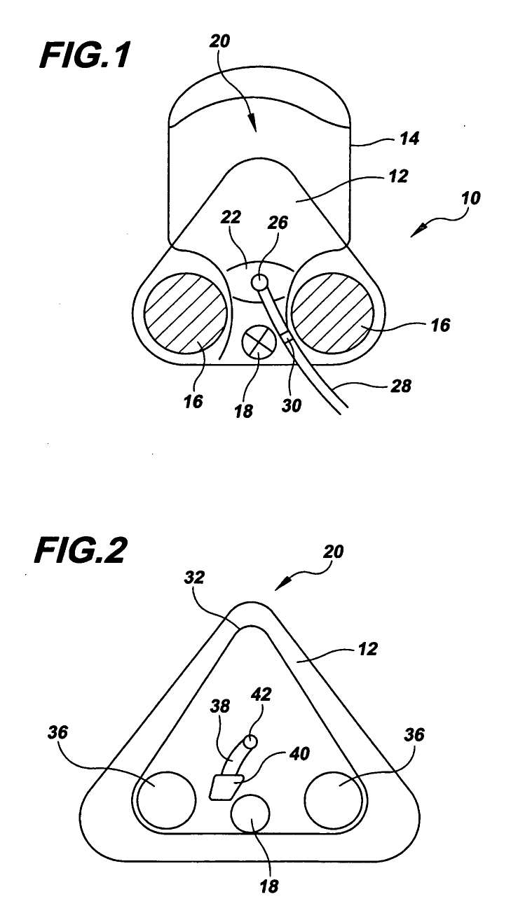

In FIG. 1, is shown a front-side view of a common half-face respirator 10. The respirator includes a face piece 12. Typically, this face piece 12 is made of a durable, yet flexible material such as silicone or vulcanized rubber. However, numerous other appropriate materials would be apparent to one skilled in the art. The respirator 10 is held to the user with one or more straps 14. In this embodiment, the strap 14 is a single piece that is simply stretched over user's head. However, in other embodiments, the strap 14 could include fasteners, and there could even be additional straps. The respirator also commonly includes two filter cartridges 16 and an exhalation valve 18. In use, the nasal piece 20 of the respirator 10 would rest on the bridge of the user's nose.

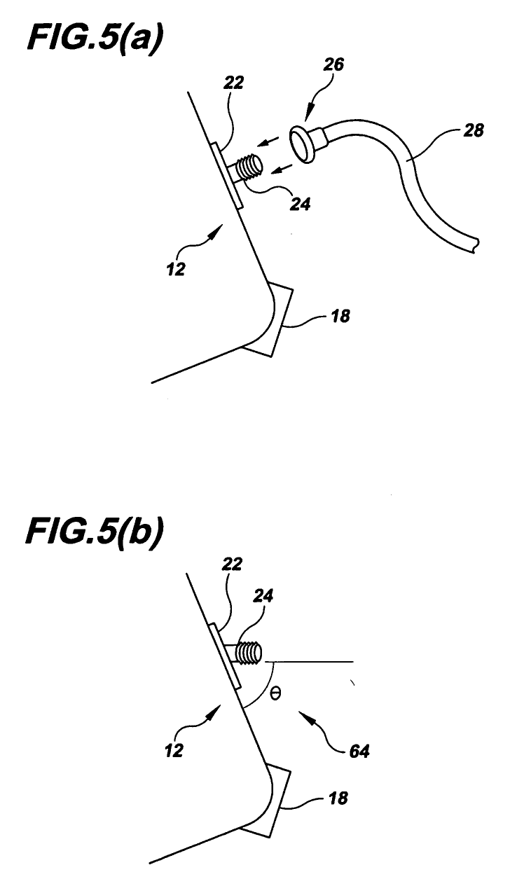

Also shown in FIG. 1 is a fluid feed-line connector base 22. This base 22 can be made of numerous materials including, but not limited to hardened rubber or plastic. As best seen in FIGS. 5(a) and 5(b), the base 22 circumscribes a fluid feed-line connector 24. In combination, the base 22 and fluid feed-line connector 24 create a substantially impervious barrier to contaminants outside the system.

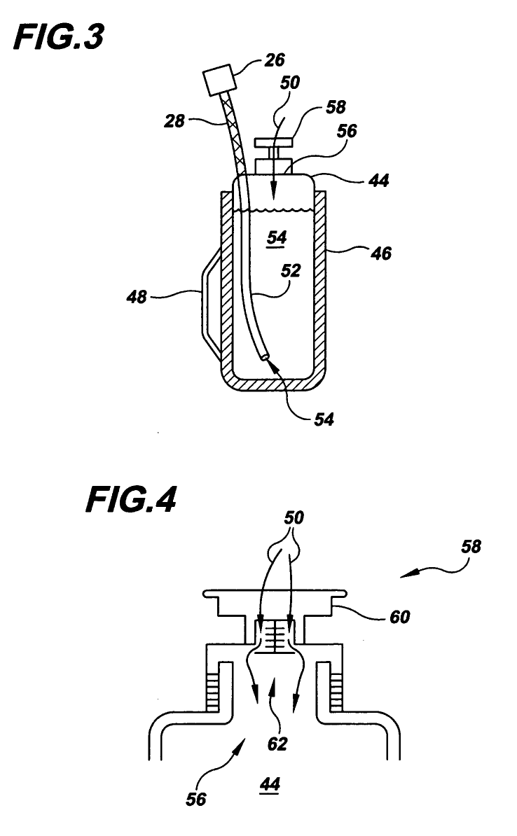

The fluid feed-line connector 24 is, in this embodiment, a threaded male fitting that is adapted to screw into a correspondingly threaded female fitting 26 on outside fluid feed-line 28. The other end of outside fluid feed-line 28 is then connected to a fluid receptacle 44 in a similar manner (as shown in FIG. 3). In the embodiment shown in FIG. 1, the outside fluid feed-line 28 can be held in place by a holder 30.

In FIG. 2 is shown an inside view of a respirator 10 equipped with a fluid feed-line attachment according to one embodiment of the present invention. As is evident from the Figure, the filter cartridges 16 correspond to the filtered air passageways 36 (i.e. the air that passes through filters 16, enters the respirator at this point). Also visible on this inside view is the exhalation valve 18, and the nasal cavity 20, showing more clearly the portion 32 of the respirator 10 the rests on the bridge of the user's nose.

The inside fluid feed-line 38 is coupled on one end to the inside of the face-piece 12 at connection point 40, and is positioned such that a user would be able to direct the second, free end of line 38 into his or her mouth when fluids were desired. In instances where it is desirable to keep all unfiltered air out of the respirator (which presumably would be most, if not all, of the time), it is important that the connection point 40 creates a substantially impervious barrier to the outside air. In this embodiment the connection point 40 could be molded as part of the feed-line 38 or could be connected with another threaded fitting as is utilized on the outside at 26.

In the present embodiment, the fluid feed-line 38 includes a bite valve 42 at the free end of the fluid feed-line 38. Thus, a user is able to open the valve 42 by clenching the feed-line in his or her teeth, thereby permitting the drawing of fluid through the line and into the user's mouth.

In FIG. 3 is shown a fluid receptacle 44 suitable for use with a fluid feed-line attachment according to one embodiment of the present invention. In this embodiment, the receptacle 44 is a standard water bottle, however, other suitable receptacles 44 include, but are not limited to camelback type bags or collapsible containers. As noted above, the outside fluid feed-line 28 connects at one end to the feed-line connector 24 on the respirator 10, through the threaded female fitting 26. At the other end, the outside fluid feed line 28 attaches to the receptacle 44. In this embodiment, the outside fluid feed-line 28 is a ¼″ surgical tube with flexible braided wire reinforcement.

In the embodiment shown in FIG. 3, the outside fluid feed line 28 continues (albeit without the metal flex tubing reinforcement) down into the bottom of the receptacle 44 as shown at 52. However, it is noted that in other embodiments, rather than being one continuous piece, the outside fluid feed line 28 can simply connect to the receptacle 44 through similar threaded or compression threaded connections as discussed above, and the portion of the tube 52 that extends down into the receptacle 44 is a separate piece. In either case, all that is necessary is that there be fluid communication between the portion of the feed-line 52 in the receptacle 44, and the portion of the feed-line 28 that connects to the respirator 10, and that the communication be substantially impervious to outside air.

The fluid receptacle 44 also can include a filter 58 that permits the user to draw fluid 54 from the receptacle without either ingesting contaminants, or creating a negative pressure in the receptacle thereby rendering the system non-operational. Specifically, as best seen in FIG. 4, the receptacle 44 can include a filter 58 over the opening 56 to the outside environment. When a user draws fluid 54 through the inside fluid feed-line 38, to balance out the negative pressure created thereby, air 50 will flow into the receptacle 44. If the surrounding air is contaminated (as is very likely the case), then, the user would effectively be contaminating the fluid supply with every drink. If, on the other hand, no air were permitted to enter the receptacle 44, then after relatively few drinks, the resultant negative pressure would prevent any further fluid from being drawn from the receptacle 44.

To address this problem, the present invention can include a filtering system 58. The filter system 58 can include a HEPA cartridge 60 through which the air 50 passes as it is being drawn into the receptacle 44. Additionally, the embodiment of FIG. 4 includes a spring valve 62 (shown in open position). Thus, when the user is drawing fluid 54 from the receptacle 44, resulting in an influx of air 50, the spring valve 62 opens permitting the passage of air 50 at that time and in that direction. However, once the user has ceased drawing fluid 54 from the receptacle 44, the valve 62 automatically closes.

FIGS. 5(a) and 5(b) simply show different orientations of the feed-line connector 24. In FIG. 5(a), the feed-line connector is oriented at a right angle to the surface of the face piece 12. In FIG. 5(b), the orientation of the connector 24 is at angle θ (shown at 64).

To illustrate the advantages of certain features of the embodiments discussed above, an operational example is given below. However, this illustration is not intended to limit the claimed invention to any particular embodiment, or method of operation.

In operation, a user would place the respirator 10 on his or her face. The fluid receptacle 44 could be attached to the user's belt through belt loop 48 (FIG. 3). The outside fluid feed-line 28 would then be attached to the feed-line connector 24 on the respirator 10 by attaching the female fitting 26 to the male feed-line connector 24.

When the user desired a drink, he or she would simply seize the inside fluid feed-line 38, on the inside of the respirator 10, with his or her tongue, lips or teeth. The user could then bite down on the bite valve 42, and by sucking, draw fluid 54 from the receptacle 44, up through the outside feed-line 28. The fluid 54 would then pass through the feed-line connector 24, into the inside feed-line 38, and from there into the user's mouth.

As the user draws fluid 54 from the receptacle 44, air 50 is pulled down into the receptacle 44 through filter 58, opening spring valve 62. Once the user has taken a drink, he or she releases the inside feed-line 38 thereby closing the bite valve 42. Because air 50 is no longer being drawn into the receptacle 44, the spring valve 62 reverts to a closed position.

Variations of the Present Invention

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes, which come within the meaning and range of equivalency of the claims, are to be embraced within their scope, and numerous modifications and alternative arrangements may be devised by those skilled in the art without departing from the spirit and scope of the present invention.

For example, FIGS. 1-5(b) primarily illustrate the present system in connection with half-faced respirators. However, the same technology could likewise be incorporated into full-face respirators, P.A.P.R., and other types of respirators including gasmasks.

Additionally, much of the discussion above is in the context of using the respirators in connection with asbestos removal. And while such a use is clearly envisioned to be within the scope of the present invention, this system could also have application in connection to firefighting, lead paint abatement, mold remediation, Hazmat situations, military or police actions and crisis situations.

Moreover, in the embodiment shown in FIGS. 1 and 5(a) and (b), the base 22 is shown circumscribing the connector 24. However, other types of connections would be suitable provided they were able to create a substantially impervious barrier to the outside air.

The mechanism whereby the outside fluid feed-line 28 is coupled to the respirator 10 is largely discussed above as a threaded connection or a compression threaded connection. However, other connection types may be suitable.

It is also noted that in embodiments utilizing threaded connections, the female fitting could be on the respirator 10, while the male fitting could be on the feed-line 28. It is also noted that the filter 58 on receptacle 44 is shown substantially at a top position of the receptacle 44. However, it is not necessary that the filter 58 be on top, but could likewise be on an upper side of the receptacle 44, provided the level of the filter 58 is such that it would not be at or below the fluid level.

The filters 58 described in the embodiments above were HEPA filters. HEPA is an acronym for “High Efficiency Particulate Air”, and is a type of filter certified to filter air particulates, such as asbestos. However, while the present invention is very well-suited for use in asbestos removal, it really could be used in any circumstances where contact with the outside air is not desirable. For example, in some circumstances, a respirator mask may be necessary to keep out toxic gases, fumes, particulate matter, or any combination thereof (e.g. asbestos, acid, formaldehyde, ammonia, mercury, or chlorine fumes, etc.). In each case, the type of filter that would be used, would depend on the type of hazardous situation being faced by the user. For example, if a poisonous gas, rather than particulate matter, was in the outside air, a different type of filter would be used—such as a charcoal filter. Numerous other types of filters would be apparent to one skilled in the art.

With respect to particular HEPA cartridges that are suitable for use with the present invention, they include, but are not limited to, HEPA filters manufactured by North Safety Products (Cranston, R.I.); 3M (St Paul Minn.); Mine Safety Appliances Company (Pittsburg, Pa.); and Moldex (Culver City, Calif.).

What is claimed is:

1) A respirator fluid feed-line system comprising:
a) a respirator having:
i) a first side and a second side opposite the first side;
i) a feed-line connector on the first side; and
ii) an inside fluid feed-line on the second side, in communication with the feed-line connector on a first end, and having a free second end capable of being inserted into a user's mouth;
b) a fluid receptacle having:
i) an opening through which air can pass; and
ii) a filter, in communication with the opening, and positioned substantially between the opening and an outside air source; and wherein the filter includes:
a HEPA cartridge; and
a spring valve, such that when air is passing into the receptacle, the spring valve is opened, and when air is not passing into the receptacle, the spring valve is closed; and
c) an outside fluid feed-line, having a third and a fourth end, wherein the third end is capable of being coupled to the feed line connector, and the fourth end is capable of being coupled to the fluid receptacle such that the fluid receptacle, the outside fluid feed-line and the inside fluid feed-line are in communication with each other.

2) The system of claim 1, further comprising:
a) a threaded female fitting on the first end of the outside fluid feed-line; and
b) a corresponding threaded male fitting on the feed-line connector.

3) The system of claim 1, wherein the inside feed-line includes a bite valve.

4) A respirator fluid feed-line system comprising:
a) a respirator having:
i) a first side and a second side opposite the first side;
i) a feed-line connector on the first side; and
ii) an inside fluid feed-line on the second side, in communication with the feed-line connector on a first end, and having a free second end that is capable of being inserted into a user's mouth;
b) a fluid receptacle; and
c) an outside fluid feed-line, having a third and a fourth end, wherein the third end is capable of being coupled to the feed line connector, and the fourth end is capable of being coupled to the fluid receptacle such that the fluid receptacle, the outside fluid feed-line and the inside fluid feed-line are in communication with each other.

5) The system of claim 4, further comprising:
a) a threaded female fitting on the third end of the outside fluid feed-line; and
b) a corresponding threaded male fitting on the feed-line connector.

6) The system of claim 4, wherein the inside feed-line includes a bite valve.

7) The system of claim 4, wherein the fluid receptacle further comprises:
a) an opening through which air can pass; and
ii) a filter, in communication with the opening, and positioned substantially between the opening and an outside air source.

8) The system of claim 7, wherein the filter includes a HEPA cartridge.

9) The system of claim 7, wherein the filter includes a spring valve, such that when air is passing into the receptacle, the spring valve is opened, and when air is not passing into the receptacle, the spring valve is closed.

10) The system of claim 1, wherein the fluid receptacle includes an insulated cover.

11) A method of providing a fluid to a user of a respirator comprising the steps of:
a) providing a respirator having:
i) a first side and a second side opposite the first side;
i) a feed-line connector on the first side; and
ii) an inside fluid feed-line on the second side, in communication with the feed-line connector on a first end, and having a free second end capable of being inserted into a user's mouth;
b) coupling a third end of an outside fluid feed-line to the feed line connector;
c) coupling a fourth end of the outside fluid feed-line to the fluid receptacle such that the fluid receptacle, the outside fluid feed-line and the inside fluid feed-line are in communication with each other;
d) inserting the free second end of the inside fluid feed-line into the user's mouth;
e) providing suction at the free second end, thereby creating a negative pressure in the inside fluid feed-line; whereby the fluid is conveyed from the receptacle, through the outside fluid feed-line, through the feed-line connector, through the inside fluid feed-line and into the user's mouth.

12) The method of claim 11, further comprising:
a) a threaded female fitting on the third end of the outside fluid feed-line; and
b) a corresponding threaded male fitting on the feed-line connector.

13) The method of claim 11, wherein the inside feed-line includes a bite valve.

14) The method of claim 11, wherein the fluid receptacle further comprises:
a) an opening through which air can pass; and
ii) a filter, in communication with the opening, and positioned substantially between the opening and an outside air source.

15) The method of claim 14, wherein the filter includes a HEPA cartridge.

16) The method of claim 14, wherein the filter includes a spring valve, such that when air is passing into the receptacle, the spring valve is opened, and when air is not passing into the receptacle, the spring valve is closed.

17) The method of claim 14, wherein the fluid receptacle includes an insulated cover.
